(19) United States
(12) Patent Application Publication
(10) Pub. No.: US 2016/0069890 A1
(43) Pub. Date: Mar. 10, 2016

(54) MOLECULAR LABELING METHODS

(71) Applicant: Bio- Techne Corporation, Minneapolis, MN (US)

(72) Inventors: Zhengliang L. Wu, Edina, MN (US); Alexander Kalyuzhny, Roseville, MN (US)

(21) Appl. No.: 14/847,549

(22) Filed: Sep. 8, 2015

Related U.S. Application Data
(60) Provisional application No. 62/047,904, filed on Sep. 9, 2014.

(51) Int. Cl. G01N 33/58; G01N 33/533; C07K 16/28; C12P 21/00; C07K 14/47; C07K 14/525

(52) U.S. Cl. CPC G01N 33/582; C07K 14/47; C07K 14/525; C07K 16/2866; C12P 21/005; G01N 33/533; C07K 2317/20; G01N 2440/38; C07K 2317/41; C07K 2317/14

(57) ABSTRACT

The disclosed methods involve deglycosylating a target molecule to remove target carbohydrates to create vacant glycosylation acceptor sites and then using glycosyltransferases to incorporate replacement carbohydrates into those sites. The methods also involve using glycosytransferases to incorporate new carbohydrates into vacant glycosylation acceptor sites without having to perform in vitro deglycosylation. The replacement or new carbohydrates include a click chemistry moiety that reacts to a click chemistry moiety on a label.

### RELATED APPLICATIONS

This application claims the benefit of U.S. Provisional Patent Application No. 62/047,904, filed Sep. 9, 2014, the entire contents of which are incorporated herein by reference. This application also relates to U.S. utility application titled “Methods for Determining Presence or Absence of Glycan Epitopes on Glycoproteins” and filed concurrently herewith under attorney document number 10805.136.1, the entire contents of which are incorporated herein by reference.

### BACKGROUND

Proteins and carbohydrates are fundamentally important in all kinds of living organisms. Labeling techniques are essential for the research of proteins and carbohydrates. However, many drawbacks exist with existing labeling techniques. For example, many labeling techniques are antibody based, which have challenges when the target molecule is not as antigenic and the antibodies have too low of affinity to their antigens. Other labeling techniques involve tagging a reporter molecule to the side chains of amino acid residues of lysine and cysteine of a protein. However, because lysine and cysteine may be essential for biological functions, the tagging may adversely affect the functionality of the protein. Other methods of labeling include N-hydroxysussinimide (NHS) based labeling and isothiocyanate based labeling, which rely on reacting to the primary amine or sulfhydryl groups on a protein. Because primary amine and sulfhydryl groups are very common on protein, it is rather difficult to achieve high level of specificity, optimal stoichiometry, and desired localization of the labeling with these methods. In fact, NHS based labeling could give labeling of any primary amine group in a protein. Other methods include enzymatic labeling methods, such as biotin ligase based biotinylation, transglutaminase based labeling, and mutant glycosyltransferase based labeling. However, these methods can only be applied to restricted number of proteins, as the acceptor sites on these proteins are rare and the numbers of such enzymes for labeling are limited.

Therefore, it would be desirable to provide new methods for labeling molecules, such as proteins and carbohydrates, that do not have as many drawbacks. In particular, it would be desirable to provide new methods for labeling molecules that are relatively quick and convenient, capable of labeling a broad range of molecules at low concentrations, capable of achieving high specificities, and are not likely to adversely affect the biological functionality of the molecules.

### SUMMARY

Improved methods and systems for in vitro labeling of target molecules are described. Glycosylation is an enzymatic process that attaches glycans to molecules. For example, proteins often form conjugates in the form of glycoproteins or proteoglycans through glycosylation. Glycosylation of protein is the most common type of post-translational modification and majority of proteins expressed by human cells are glycosylated. Since proteins are frequently glycosylated, in many cases, the conjugated glycans are dispensable for the biological functions of the proteins. The biological activities of many proteins may not be detectably different if some of the glycans or carbohydrate residues on the proteins are removed or replaced. The current methods involve deglycosylating a target molecule to remove target carbohydrates to create vacant glycosylation acceptor sites and then using glycosyltransferases to incorporate replacement carbohydrates at those sites. The methods also involve using glycosyltransferases to incorporate new carbohydrates into vacant glycosylation acceptor sites without having to perform in vitro deglycosylation. The replacement carbohydrates or new carbohydrates include a click chemistry moiety that reacts to a click chemistry moiety on a label. The disclosed methods have several advantages over existing labeling techniques. For example, some embodiments increase the ease and speed of labeling or modification, can be applied to a broad range of target molecules, increase the specificity of the labeling or modification, and unlikely adversely affect the biological functionality of the target molecules.

Some embodiments provide an in vitro method of labeling a target molecule (e.g., a target glycoprotein), comprising: (a) providing a sample containing a target molecule, (b) treating the sample with a glycosidase to remove a target carbohydrate on the target molecule, thereby creating a vacant glycosylation acceptor site on the target molecule, wherein the glycosidase is specific for the target carbohydrate, (c) treating the sample with a glycosyltransferase to incorporate a replacement carbohydrate into the vacant glycosylation acceptor site, wherein the glycosyltransferase has a substrate specificity that matches or overlaps with a substrate specificity of the glycosidase and wherein the replacement carbohydrate includes a click chemistry moiety; and (d) adding a label to the sample, wherein the label includes a click chemistry moiety that reacts to the click chemistry moiety of the replacement carbohydrate such that the label attaches to the replacement carbohydrate.

Other embodiments provide an in vitro method of labeling a target molecule (e.g., a target glycoprotein), comprising: (a) providing a sample containing a target molecule that has a vacant glycosylation acceptor site existing without having to perform in vitro deglycosylation, (b) treating the sample with a glycosyltransferase to incorporate a new carbohydrate into the vacant glycosylation acceptor site, wherein the glycosyltransferase is specific for the vacant glycosylation acceptor site and wherein the replacement carbohydrate includes a click chemistry moiety, and (c) adding a label to the sample, wherein the label includes a click chemistry moiety that reacts to the click chemistry moiety of the replacement carbohydrate such that the label attaches to the replacement carbohydrate.

Other embodiments provide an in vitro method of labeling target glycoproteins, comprising: (a) providing a sample containing target glycoproteins, (b) performing a deglycosylation step to remove target carbohydrates on the target glycoproteins, thereby creating a vacant glycosylation acceptor sites on the target glycoproteins, wherein the deglycosylation is specific for the target carbohydrates, (c) performing glycosylation to incorporate replacement carbohydrates into the vacant glycosylation acceptor site on the target glycoproteins, wherein the glycosylation is specific for the vacant glycosylation acceptor site and wherein the replacement carbohydrates include a click chemistry moiety that can be used in a click chemistry reaction, and (d) performing a click chemistry reaction to attach labels to the replacement carbohydrates, wherein the labels include a click chemistry moiety that reacts to the click chemistry moiety of the replacement carbohydrates such that the labels attach to the replacement carbohydrates.

Yet other embodiments provide an in vitro method of conjugating a glycoprotein to another molecule, comprising: (a) providing a sample containing a glycoprotein, (b) treating the sample with glycosidase to remove a target carbohydrate on the glycoprotein, thereby creating a vacant glycosylation acceptor site on the glycoprotein, (c) treating the sample with a glycosyltransferase to incorporate a replacement carbohydrate into the vacant glycosylation acceptor site, wherein the replacement carbohydrate includes a click chemistry moiety, and (d) adding a molecule to the sample, wherein the molecule includes a click chemistry moiety that reacts to the click chemistry moiety of the replacement carbohydrate such that the molecule attaches to the replacement carbohydrate. The clickable molecule can include an antibody or a drug or another molecule.

In each of the methods, the replacement carbohydrate can be a sialic acid, GalNAc, GlcNAc, fucose or galactose in some embodiments. Further, in some cases the replacement carbohydrate can include a click chemistry moiety selected from one of an azido group or an alkyne group and the label can include a click chemistry moiety selected from the other of the azido group or an alkyne group. Also, in some cases the label can include a label selected from a biotin molecule, a fluorogenic or fluorescent molecule or a luminescent molecule. The fluorogenic molecule can be a derivative of fluorescein or rhodamin in some cases. Also, the luminescent molecule can be a luminol in some cases.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a flow diagram showing a method of labeling a target molecule according to various embodiments.

FIG. 2 is a flow diagram showing another method of labeling a target molecule according to various embodiments.

FIG. 3 is a flow diagram showing a method of modifying a target molecule according to various embodiments.

FIG. 4 is a representation of labeling a target glycoprotein through sialic acid replacement according to various embodiments.

FIG. 5 shows images of the results of labeling a target glycoprotein through sialic acid replacement according to various embodiments. “F” stands for fetuin and “DF” stands for desialylfetuin.

FIG. 6 shows images of the results of labeling a target glycoprotein through sialic acid replacement according to various embodiments (left side) and direct labeling of a target glycoprotein according to various embodiments (right side).

FIG. 7 is a representation of labeling a target glycoprotein through O-glycan replacement according to various embodiments.

FIG. 8 shows images of the results of labeling a target glycoprotein through O-glycan replacement according to various embodiments.

FIG. 9 shows images of the results of labeling a target glycoprotein through direct labeling according to various embodiments.

FIG. 10 shows images of the results of labeling a target glycoprotein through direct labeling according to various embodiments.

### DETAILED DESCRIPTION

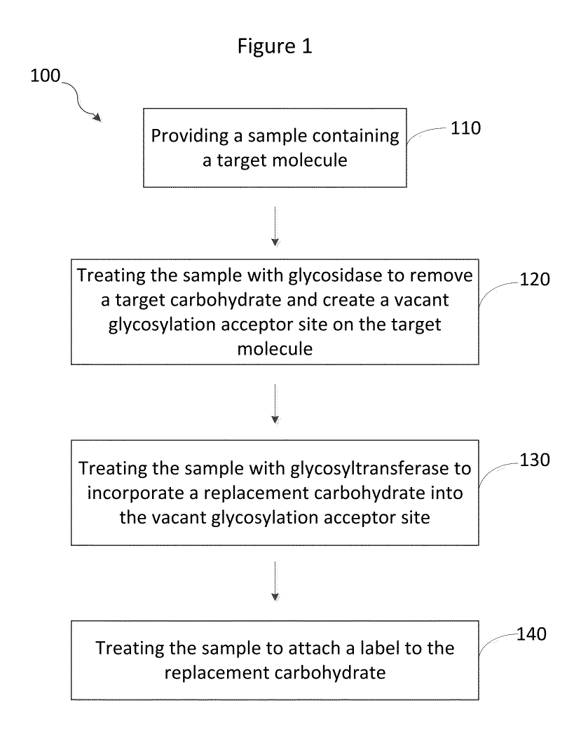

Some embodiments provide in vitro methods of labeling a target molecule. A first in vitro method 100 is represented through the flow chart shown in FIG. 1. The method 100 generally includes providing a sample containing a target molecule at step 110, treating the sample with a glycosidase to remove a target carbohydrate on the target molecule, thereby creating a vacant glycosylation acceptor site on the target molecule at step 120, treating the sample with a glycosyltransferase to incorporate a replacement carbohydrate into the vacant glycosylation acceptor site at step 130, and treating the sample to attach a label to the replacement carbohydrate at step 140. Each of these steps will now be discussed in more detail.

The method 100 includes a step 110 of providing a sample containing a target molecule. The target molecule can include any molecule having a target carbohydrate that occupies a glycosylation acceptor site. In other words, the target molecule can be any molecule that is glycosylated, such as glycosylated proteins or glycosylated lipids. In some cases, the target molecule is a glycoprotein. In certain cases, the target glycoprotein is a glycoprotein selected from secreted glycoproteins, membrane glycoproteins or intracellular glycoproteins. In some cases, the target glycoprotein is an O-GlcNAcylated protein.

The target molecule can be provided in a solution, for example a buffer solution. In some cases, the target molecule is a target glycoprotein provided in a buffer solution. Suitable buffer solutions include Tris, HEPES, MES or any other kind of Good's buffer solutions.

The method also includes a step 120 of treating the sample with a glycosidase to remove a target carbohydrate and create a vacant glycosylation acceptor site on the target molecule. Possible target carbohydrates that can be removed by a glycosidase include sialic acid, fucose, GlcNAc, GalNAc, galactose, mannose and xylose. Possible glycosidase that can be used include sialidase, fucosidase, hexosaminidase and galactosidase. In some cases, the glycosidase is a sialidase and the target carbohydrate is removed by sialidase treatment.

In some cases, the treating the sample with a glycosidase comprises mixing the sample with a buffer containing a glycosidase for a period of time and at a temperature and then removing or inactivating the glycosidase after the period of time. In certain cases, the removing the glycosidase comprises separating the glycosidase from the sample on a chromatograph column. In other cases, the inactivating the glycosidase comprises heating the sample to a temperature for a period of time. In some cases, the temperature is a temperature in the range of between 55° C. and 98° C. Also, in some cases, the period of time is a period in the range of between 2 to 10 minutes.

In some embodiments, the target carbohydrate that is removed is a terminal monosaccharide, such as a terminal sialic acid. In other embodiments, the target carbohydrate is an oligosaccharide, such as an O-glycan. In some cases, an O-glycan is removed and is one selected from Core-1, Core-2, Core-3, Core-4, Core-5, Core-6, Core-7 and Core-8 O-glycans. Once the target carbohydrate residue is removed, the target protein has a vacant glycosylation acceptor site where the target carbohydrate used to be.

The glycosidase a specific glycosidase or a combination of specific glycosidases that specifically removes the target carbohydrate. In some cases, the target carbohydrate is a monosaccharide and is removed by a single glycosidase. In other cases, the target carbohydrate is an oligosaccharide and is removed by a combination of glycosidases. For example, Thomsene-Friedenreich antigen and its sialyl version (sialyl-T antigen) are two O-glycans known to be present on bovine serum fetuin that can be removed with the endoglycosidase E. faecalis Endo-EF and recombinant C. perfringens neuraminidase. Other various O-glycans can be removed with a combination of a galactosidase or an endoglycosidase E. faecalis Endo-EF and C. perfringens neuraminidase.

The method also includes a step 130 of treating the sample with a glycosyltransferase to incorporate a replacement carbohydrate at the vacant glycosylation acceptor site. The replacement carbohydrate includes a click chemistry moiety that can be used in a click chemistry reaction, such as an azido or an alkyne group. In some cases, the replacement carbohydrate is a monosaccharide. The glycosyltransferases used to incorporate replacement carbohydrates into the vacant glycosylation acceptor sites can include but are not limited to sialyltransferases, fucotransferases, GlcNAc transferases, GalNAc transferases, galactosyltransferases, glucosyltransferases, xylosyltransferases and mannosyltransferases.

The method further includes a step 140 of adding a label to the sample so that a click chemistry reaction is performed. Click chemistry is a way to quickly and reliably join small units together. It is not a single specific reaction, but refers to a general way of joining small modular units. The label includes a click chemistry moiety that reacts to the click chemistry moiety of the replacement carbohydrate such that the label attaches to the replacement carbohydrate. In some cases, the replacement carbohydrate includes an azido group and the label includes an alkyne group. In other cases, the replacement carbohydrate includes an alkyne group and the label includes an azido group. The clickable label can be a reporter molecule such as a colorimetric label, a biotin label, a luminescent label or a fluorescent label.

In the method 100, a high level of specificity can be achieved by two levels of enzymatic reaction. The first level is by the selection of a glycosidase that removes a target carbohydrate to create a vacant glycosylation acceptor site. The second level is by the selection of a glycosyltransferase that only recognizes the specific vacant glycosylation acceptor site. Also, the glycosidase has a substrate specificity that matches or overlaps with a substrate specificity of the glycosyltransferase. In some cases, the glycosidase is a sialidase and the glycosyltransferase is a sialyltransferase, wherein the substrate specificity of the sialidase matches or overlaps with that of the sialyltransferase. For example, a sialidase specific for 6-O sialic acid may be used in pair with a 6-O specific sialyltransferase. Following are exemplary glycosidase-glycosyltransferase pairs that can be used to replace certain monosaccharides:

TABLE

| Monosaccharides | | |
|---|---|---|
| that are | | |
| replaced | Glycosidases | Glycosyltransferases |
| Sialic acid | α2-3 specific sialidases | STGal1, 2, 3, 4, 5, |
| | α2-6 specific sialidases | ST6Gal1, 2, |
| | α2-8 specific sialidases | ST6GalNAc1, 2, 3, 4, 5, 6 |
| | | ST8Sia1, 2, 3, 4. |
| fucose | α-fucosidase | FUT1, 2, 3, 4, 5, 6, 7, 8, 9, |
| | | 10, 11 |
| GlcNAc | α-hexosaminidases | MGAT1, 2, 3, 4, 5 |
| | β-hexosaminidases | GCNT1, 2, 3, 4, 5, 6 |
| | | B3GNT1, 2, 3, 4, 5, 6, 7 |
| | | A4GNT |
| GalNAc | α-hexosaminidases | GALNT1, 2, 3 . . . to 20 |
| | β-hexosaminidases | |
| T antigen | O-glycosidase | GALNT1, 2, 3 . . . to 20 |
| | (Endo EF) | |

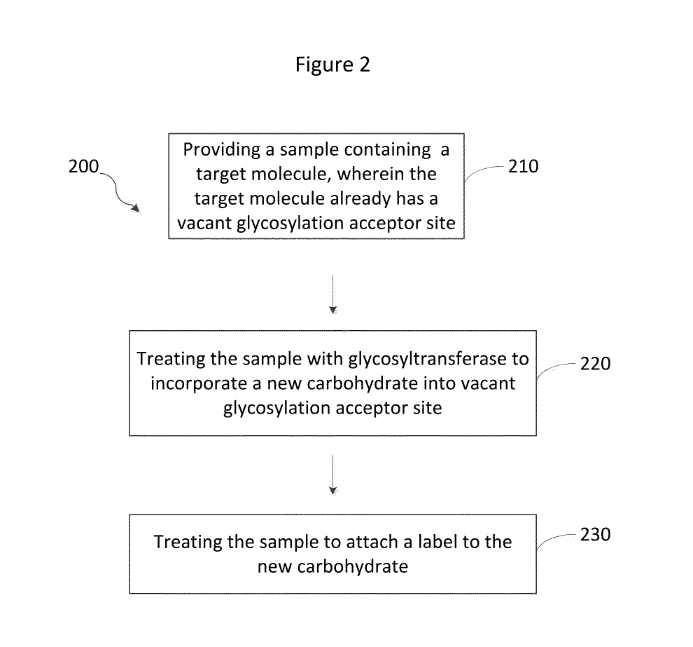

Another in vitro method 200 of labeling a target molecule is represented through the flow chart shown in FIG. 2. The method 200 includes providing a sample containing a target molecule at step 210, wherein the target molecule has a vacant glycosylation acceptor site existing without having to perform in vitro deglycoslylation, treating the sample with a glycosyltransferase to incorporate a new carbohydrate into the vacant glycosylation acceptor site at step 220, and adding a label to the sample, wherein the label attaches to the new carbohydrate at step 230. The second method 200 differs from the first method 100 in that the target molecule already has a specific vacant glycosylation acceptor site and thus a step of removing a target carbohydrate residue with a glycosidase to create a vacant glycosylation acceptor site is not needed. For example, some glycoproteins are purified from sources that are devoid of glycosylation machinery such as E. coli sources. These glycoproteins already have vacant glycosylation acceptor sites so deglycosylation may not be necessary. In the method 200, a high level of specificity can be achieved by selection of a glycosyltransferase that only recognizes one of the existing vacant glycosylation acceptor sites.

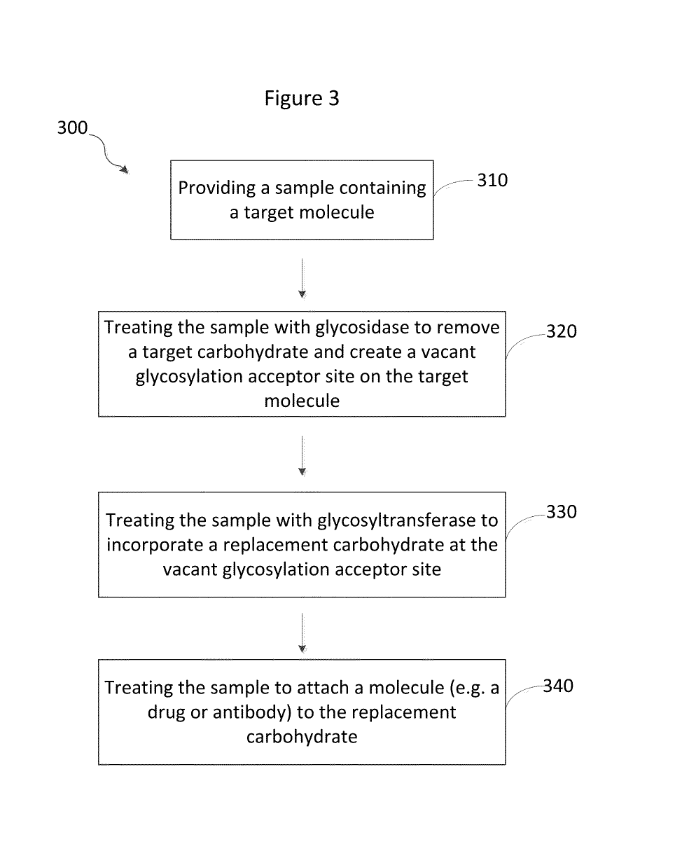

Some embodiments provide in vitro methods of conjugating target molecules to other molecules. For example, one exemplary conjugating method 300 is represented through the flow chart shown in FIG. 3. The method 300 generally includes a step 310 of providing a sample containing a target molecule, a step 320 of treating the sample with a glycosidase to remove a target carbohydrate on the target molecule, thereby creating a vacant glycosylation acceptor site on the target molecule, wherein the glycosidase is specific for the target carbohydrate, a step 330 of treating the sample with a glycosyltransferase to incorporate a replacement carbohydrate into the vacant glycosylation acceptor site, and a step 340 of adding a molecule to the sample. In this method, the replacement carbohydrate includes a click chemistry moiety and the molecule includes a click chemistry moiety that reacts to the click chemistry moiety of the replacement carbohydrate such that the molecule attaches to the replacement carbohydrate. The molecule can be any other desired molecule that attaches to the replacement carbohydrate. For example, the molecule can be a drug, antibody, lipid or protein in some embodiments.

### EXPERIMENTAL

### Materials

For each of Examples 1-3, bovine serum fetuin and CTP were obtained from Sigma Aldrich. Recombinant human enzymes ST3Gal1, ST3Gal5, ST6Gal1, GALNT1, GALNT2, GALNT3, GALNT7, CD73, CD39L3, recombinant human TNFα and TNFβ, biointylated recombinant TNFα, recombinant yeast pyrophosphatase S. cerevisiae PPA1, recombinant N. meningitidis SiaB, recombinant E. faecalis Endo-EF, recombinant C. perfringens neuraminidase, mouse anti-human IL1 IgG and its biotinylated version, streptavidin conjugated horseradish peroxidase (strep-HRP) and its enhanced chemiluminescence (ECL) substrate were obtained from Bio-Techne/R&D Systems. Click-IT biotin DIBO alkyne was obtained from Life Technologies. CMP-azido sialic acid, UDP-azido-GalNAc and a biotin alkyne adduct D were synthesized at Bio-Techne.

CMP-azido-sialic acid was synthesized as follows. 2 μmol CTP plus 2 μmol 9-azido sialic acid were mixed with 20 μg of recombinant N. meningitidis Sia B and 5 μg of recombinant yeast pyrophosphatase S. cerevisiae PPA1 in 0.5 mL of buffer of 25 mM Tris and 10 mM MgCl₂ at pH 7.5. The mixture was incubated at 37° C. for 1 hour to form CMP-azido-sialic acid.

UDP-azido-GalNAc was synthesized as follows. Uridine 5′-(trihydrogen diphosphate), 2′-deoxy-, P′-[2-[(2-azidoacetyl)amino]-2-deoxy-α-D-galactopyranosyl] ester ammonium salt was synthesized. Thus α-galactosamine 1-phosphate was treated with NHS-2-azidoacetate and the crude intermediate, after conversion to the triethylammonium salt, was reacted directly with uridine 5′-monophosphomorpholidate 4-morpholine-N,N-dicyclohexylcarboxamidinesalt. Crude purification using HILIC preparative chromatography and lyophilization gave a mixture containing 66% of uridine 5′-(trihydrogen diphosphate), 2′-deoxy-, P′-[2-[(2-azidoacetyl)amino]-2-deoxy-α-D-galactopyranosyl] ester ammonium salt.

Biotin alkyne adduct D 3-(2′-(2″-(2′″-Amide-D-biotin-ethoxy)ethoxy)ethoxy) prop-1-yne was synthesized as follows. 2-(2-(2-(prop-2-yn-1-yloxy)ethoxy)ethoxy)ethanamine was synthesized and converted into Biotin alkyne adduct D 3-(2′-(2″-(2′″-Amide-D-biotin-ethoxy)ethoxy)ethoxy) prop-1-yne via reaction with biotin.

### Methods

### Generating Glycosyltranferase Acceptor Sites Through Glycosidase Treatment

To remove terminal sialic acid, a glycoprotein sample was mixed with recombinant C. perfringens neuraminidase at a mass ratio of 100:1 in a buffer of 25 mM Tris, 150 mM NaCl at pH 7.5 and held at room temperature for 20 minutes. The sample was then separated on a gel filtration column to remove the recombinant C. perfringens neuraminidase. The sample then contained glycoprotein having removed sialic acid and thus vacant glycosylation acceptor sites.

To remove O-glycan, a glycoprotein sample was mixed with both recombinant C. perfringens neuraminidase and recombinant E. faecalis Endo-EF at a mass ratio of 100:1 in a buffer of 25 mM Tris, 150 mM NaCl at pH 7.5 and held at room temperature for 20 minutes. The sample was then heat treated at 95° C. for 2 minutes to inactivate the recombinant C. perfringens neuraminidase and recombinant E. faecalis Endo-EF. The sample then contained glycoprotein having removed O-glycan and thus vacant glycosylation acceptor sites.

### Glycosyltransferase Labeling Reaction

To incorporate clickable sialic acid into vacant glycosylation acceptor sites, 10 μg of a glycoprotein sample was mixed with 0.3 nmol of CMP-azido-sialic acid, 2 μg of sialyltransferase and 0.1 μg of CD73 in 50 μL of 25 mM Tris supplemented with 10 mM of MnCl₂ and 150 mM NaCl at pH 7.5 and incubated at 37° C. for a minimum of 20 minutes. CD73 hydrolyzes the by-product CMP and therefore removes any product inhibition caused by CMP.

To incorporate clickable O-glycan into vacant glycosylation acceptor sites, 10 μg of a glycoprotein sample was mixed with 0.3 nmol of UDP-azido-GalNAc, 2 μg of a ppGalNAcT (or GALNT) and 0.1 μg rhCD39L3 in 50 μL of 25 mM Tris supplemented with 10 mM of MnCl₂ and 150 mM NaCl at pH 7.5 and incubated at 37° C. for a minimum of 20 minutes. CD39L3 hydrolyzes the by-product UCP and therefore removes any product inhibition caused by UDP.

### Click Chemistry Reaction

For click chemistry reactions, ascorbic acid, CuCl₂ and biotin alkyne adduct D were directly added to the glycosyltransferase reaction having a final concentration of 2 mM, 0.1 mM and 0.1 mM respectively. The mixture was incubated at room temperature for a minimum of 30 minutes. For copper-free click chemistry reactions, 30 μM of Click-IT® biotin DIBO alkyne was added into each reaction and incubated at room temperature for 5 hours.

### Methods Used to Analyze Samples

After the click chemistry reactions, the samples were separated on 12% SDS-PAGE gel. The gels were visualized with UV in the presence of trichlorethanol (TCE staining), which reacts with an indole ring of a tryptophan amino acid of the proteins. Next, the gels were blotted to nitrocellulose paper under 25 volts for 30 minutes. The blots were then blocked with 10% fat-free milk for 10 minutes, followed by probing with strep-HRP at 30 ng/mL for 30 minutes. The blots were then washed three times with TBST buffer containing 25 mM Tris, pH 7.6, 137 mM NaCl and 0.01% Tween (TBS) for 30 minutes. The membrane was finally visualized with enhanced chemiluminescence (ECL) peroxidase substrate.

### Example 1

### Labeling Glycoprotein Through Sialic Acid Replacement

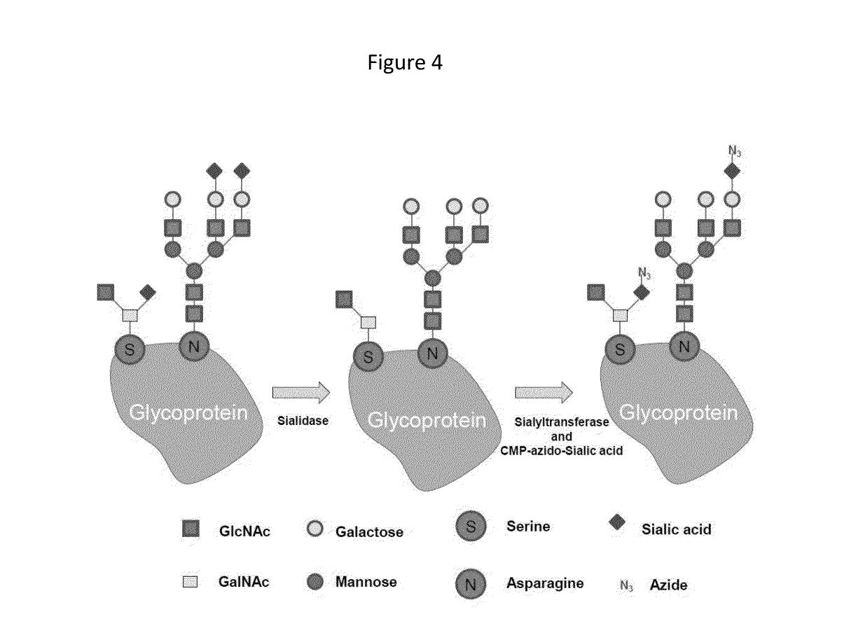

Sialic acids are usually located at non-reducing ends of glycans on various secreted glycoproteins. They regulate the solubility, stability and half-lives of secreted proteins. As shown generally in FIG. 4, terminal sialic acids are removed by sialidase treatment and replaced with clickable sialic acids using specific sialyltransferases. The specificity of the sialidase should match or overlap with that of the sialytransferase. For example, a sialidase that has a specificity for 3-O sialic acid is needed for 3-O specific sialytransferase labeling.

As an example, a sample of fetal bovine fetuin was first desialyated with recombinant C. perfringens neuramidase to generate desialylated fetuin. Separate samples of untreated fetal bovine fetuin and desialylated fetal bovine fetuin were then labeled with 9-azido sialic acid using recombinant human sialyltransferases ST3Gal1, ST3Gal5 and ST6GalNAc4 2. After labeling, the samples were conjugated to biotin alkyne adduct D via azide-alkyne Huisgen cycloaddition and then separated on SDS-PAGE. The gel was then blotted to a membrane and detected with streptavidin-conjugated horseradish peroxidase (strep-HRP).

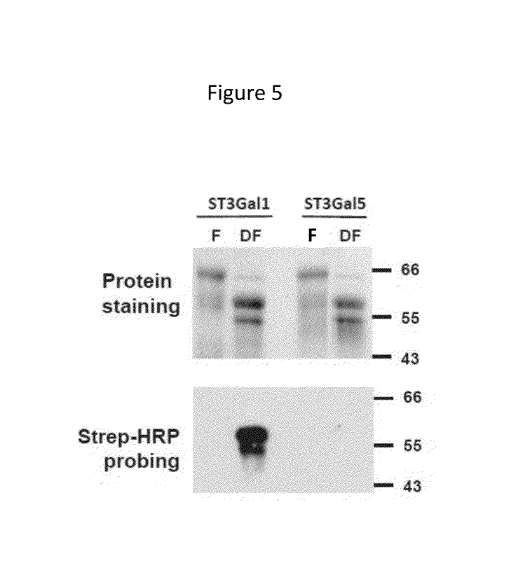

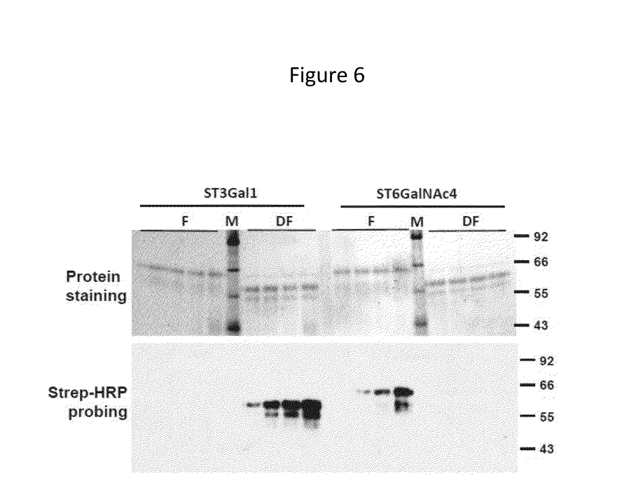

The specific results of this example are shown in the images in FIGS. 5 and 6. In each figure, the upper panels are protein staining and the lower panels are revealed with strep-HRP. As shown in FIG. 5, labeling was observed on desialylated fetal bovine fetuin samples (represented as “DF” on FIG. 5) by ST3Gal1, but not by ST3Gal5, indicating the existence of acceptor sites on DF for ST3Gal1 but not for ST3Gal5. This result is consistent with the substrate specificity of the two enzymes, i.e. ST3Gal1 sialylates core 1 type of 0-glycans and ST3Gal5 is a ganglioside specific sialyltransferase. Also, labeling was not observed on untreated fetal bovine fetuin samples (represented as “F” on FIG. 5) with ST3Gal1, suggesting that the F sample was fully α 2,3-sialylated and likely contained sialyl-core 1 O-glycan.

Since it is known that ST6GalNAc4 can add sialic acid to sialy-core 1 O-glycan and generate the disialylated tetrasaccharide Sia α 2-3Galβ1-3(Sia α 2-6) GalNAc, to prove the existence of sialyl-core 1 O-glycan and demonstrate the specificity of the labeling, the samples were further labeled with ST6GalNAc4. Indeed, as shown in FIG. 6, in contrast to ST3Gal1, ST6GalNAc4 only labeled F but not DF. Further tests on the sensitivity of ST3Gal1 labeling showed that as low as 50 ng of asialofetuin can be detected by this method and as little as 5 ng of ST3Gal1 is needed for the labeling.

### Example 2

### Labeling Glycoprotein Through 0-Glycan Replacement

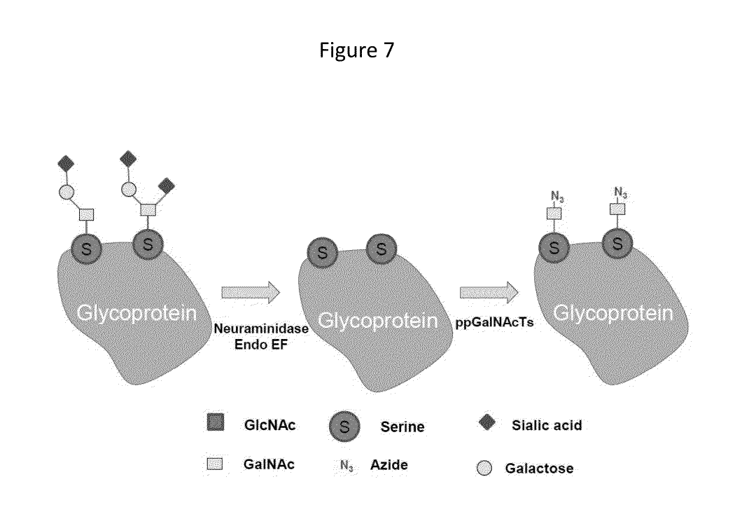

O-glycans are common glycans found on various secreted glycoproteins. They are usually relatively short oligosaccharides that can be removed with either specific glycosidases or a combination of glycosidases, and subsequently may be replaced with clickable GalNAc residues. For example, Thomsene-Friedenreich antigen (T antigen) and its sialyl version (sialyl-T antigen) are two O-glycans known to be present on bovine serum fetuin that may be removed with E. faecalis Endo-EF and recombinant C. perfringens neuraminidase and replaced with azido GalNAc residues. As shown generally in FIG. 7, E. faecalis Endo-EF and recombinant C. perfringens neuraminidase together remove the disaccharide Galβ1-3GalAc plus its sialyated versions from a glycoprotein. Subsequently, a GALNT (ppGalNAcT) adds azido-GalNAc back to the vacant glycosylation acceptor sites and therefore reveals the presents of these oligosaccharides. Other glycosidases may be used to remove other types of O-glycan.

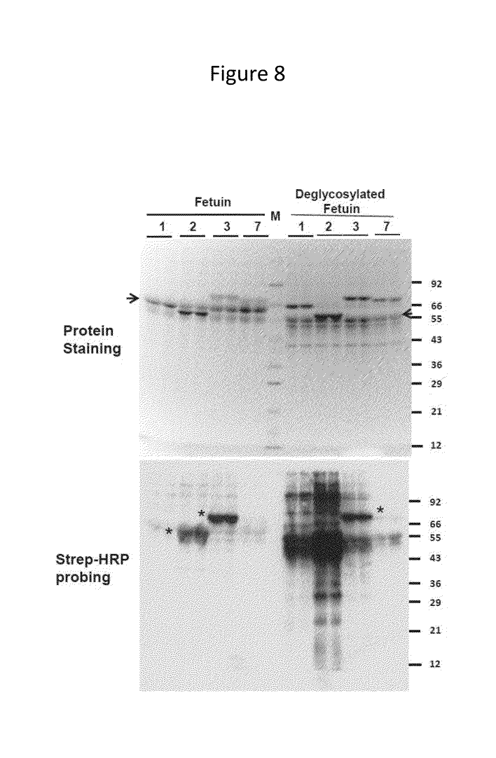

As an example, bovine serum fetuin samples were first treated with C. perfringens neuraminidase and E. faecalis Endo-EF to remove O-glycan, and then tagged with azido-GalNAc using various recombinant polypeptide GalNAc transferases (ppGALNTs or GALNTs). The specific results are shown in FIG. 8.

Left of the molecular marker M, bovine serum fetuin samples were directly labeled with GALNTs without prior deglycosylation. Right of molecular marker, fetuin samples were deglycosylated with C. perfringens neuraminidase and E. faecalis Endo-EF prior to the labeling. All samples were separated on SDS-PAGE and transferred to nitrocellulose membrane and then revealed with protein staining (upper panel) or strep-HRP probing (lower panel).

Generally, the labeling on untreated fetuin was minimal, whilst the deglycosylated fetuin was strongly labeled by GALNT1 and GALNT2, to some degree by GALNT3 but almost not by GALNT7, which is consistent with their respective specificities, i.e. GALNT1 and GALNT2 belong to early transferases that initiates glycosylation; GALNT3 is an intermediate transferase that follows glycosylation with early transferases; GALNT7 has unique substrate specificity for partial GalNAc-glycosylated acceptor substrates.

### Example 3

### Direct Labeling of Proteins or Glycoproteins that Already Contain Vacant Glycosylation Acceptor Sites

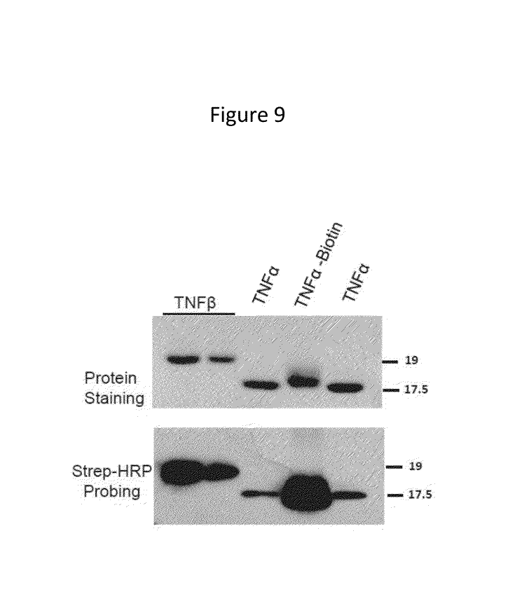

In cases where proteins are purified from a source that lacks or has defective glycosylation machinery, such as E. coli, the glycosylation sites of the expressed proteins may not be occupied, which allows for direct labeling without prior in vitro deglycosylation. In a first case for direct labeling, E. coli expressed recombinant human TNFα and TNFβ were directly treated with azido-GalNAc using GALNT2. TNFα is reported to have an O-glycosylation site at Ser-80, while TNFβ is generally reported to contain O-glycans. For comparison, a sample of TNFα that was biotinylated using NHS was run side-by-side with samples of TNFα that were labeled with the current click chemistry method. The NHS-labeled sample ran slightly slower than the click-chemistry-labeled sample, suggesting the incorporation of multiple copies of biotin in the NHS-labeled sample. Accordingly, as shown in FIG. 9, when the samples were probed with strep-HRP, the NHS-labeled sample indeed exhibited a much stronger signal than the click-chemistry-labeled samples. On the other side, the signal for click-chemistry-labeled TNFβ was stronger than that for TNFα, suggesting that TNFβ may have multiple O-glycosylation sites.

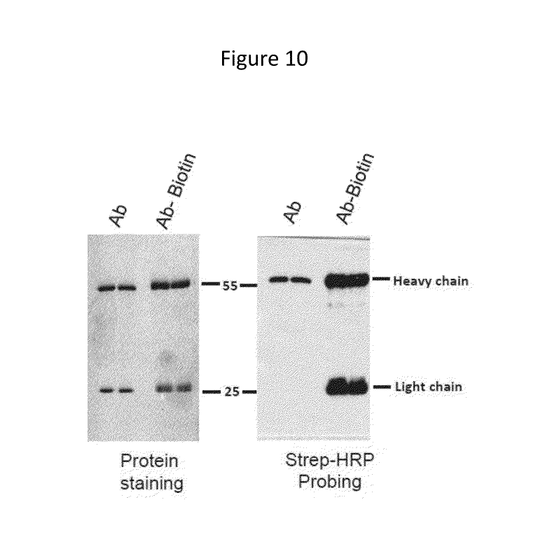

In a second case for direct labeling, a monoclonal antibody was tested. The heavy chains of antibodies are known to contain N-glycans that are low on sialylation and therefore may allow direct incorporation of azido sialic acid. To test this idea, a monoclonal mouse anti-human IL1 IgG expressed in HEK293 cell was treated with CMP-azido-sialic acid using ST6Gal1 that was known to be specific for N-glycans. The sample was then conjugated to Click-IT® biotin DIBO alkyne through a copper-free click chemistry reaction. Indeed, as shown in FIG. 10, when the labeled antibody was probed with strep-HRP, only the heavy chain appeared. In contrast, when an antibody biotinylated using NHS was probed, both the light and heavy chains showed up. This experiment not only proved the concept of direct labeling, but also again clearly demonstrated the superior specificity of the present methods.

Various examples have been described. These and other examples are within the scope of the following claims.

1. An in vitro method of labeling a target molecule, comprising:
providing a sample containing a target molecule;
treating the sample with a glycosidase to remove a target carbohydrate on the target molecule, thereby creating a vacant glycosylation acceptor site on the target molecule, wherein the glycosidase is specific for the target carbohydrate;
treating the sample with a glycosyltransferase to incorporate a replacement carbohydrate into the vacant glycosylation acceptor site, wherein the glycosyltransferase has a substrate specificity that matches or overlaps with a substrate specificity of the glycosidase and wherein the replacement carbohydrate includes a click chemistry moiety; and
adding a label to the sample, wherein the label includes a click chemistry moiety that reacts to the click chemistry moiety of the replacement carbohydrate such that the label attaches to the replacement carbohydrate.

2. The method of claim 1 wherein the target molecule is a target glycoprotein with a target carbohydrate that can be replaced with a replacement carbohydrate.

3. The method of claim 2 wherein the target carbohydrate is a target carbohydrate selected from the group consisting of sialic acid, fucose, GlcNAc, GalNAc, galactose, mannose and xylose.

4. The method of claim 1 wherein the glycosidase is a glycosidase selected from the group consisting of sialidase, fucosidase, hexosaminidase and galactosidase or combinations thereof.

5. The method of claim 1 wherein the glycosyltransferase is a glycosyltransferase selected from the group consisting of sialyltransferases, fucosyltransferases, GlcNAc transferases, GalNAc transferases, galactosyltransferases, glucosyltransferases, xylosyltransferases, mannosyltransferases and combinations thereof.

6. The method of claim 1 wherein the replacement carbohydrate includes a click chemistry moiety selected from one of an azido group or an alkyne group and the label includes a click chemistry moiety selected from the other of the azido group or the alkyne group.

7. The method of claim 1 wherein the label includes a biotin molecule, a fluorogenic molecule, or a luminescent molecule.

8. An in vitro method of labeling a target molecule, comprising:
providing a sample containing a target molecule that has a vacant glycosylation acceptor site existing without having to perform in vitro deglycosylation;
treating the sample with a glycosyltransferase to incorporate a new carbohydrate into the vacant glycosylation acceptor site, wherein the glycosyltransferase is specific for the vacant glycosylation acceptor site and wherein the new carbohydrate includes a click chemistry moiety;
adding a label to the sample, wherein the label includes a click chemistry moiety that reacts to the click chemistry moiety of the new carbohydrate such that the label attaches to the new carbohydrate.

9. The method of claim 8 wherein the target molecule is a target glycoprotein.

10. The method of claim 8 wherein the new carbohydrate is a sialic acid.

11. The method of claim 8 wherein the new carbohydrate is a GalNAc.

12. The method of claim 8 wherein the new carbohydrate includes a chemistry moiety selected from one of an azido group or an alkyne group and the label includes a click chemistry moiety selected from the other of the azido group or the alkyne group.

13. The method of claim 8 wherein the label includes a biotin molecule, a fluorogenic molecule or a luminescent molecule.

14. An in vitro method of labeling target glycoproteins, comprising:
providing a sample containing target glycoproteins;
performing deglycosylation to remove target carbohydrates on the target glycoproteins, thereby creating a vacant glycosylation acceptor sites on the target glycoproteins, wherein the deglycosylation is specific for the target carbohydrates;
performing glycosylation to incorporate replacement carbohydrates into the vacant glycosylation acceptor sites on the target glycoproteins, wherein the glycosylation is specific for the vacant glycosylation acceptor sites, wherein the replacement carbohydrates include a click chemistry moiety that can be used in a click chemistry reaction;
performing a click chemistry reaction to attach labels to the replacement carbohydrates, wherein the labels include a click chemistry moiety that reacts the click chemistry moiety of the replacement carbohydrates such that the labels attach to the replacement carbohydrates.

15. The method of claim 14 wherein the replacement carbohydrates are sialic acids.

16. The method of claim 14 wherein the replacement carbohydrates are GalNAcs.

17. The method of claim 14 wherein the replacement carbohydrates include a chemistry moiety selected from one of an azido group or an alkyne group and the labels include a click chemistry moiety selected from the other of the azido group or the alkyne group.

18. The method of claim 14 wherein the labels include a label selected from a biotin molecule, a fluorogenic molecule or a luminescent molecule.

19. An in vitro method of conjugating a glycoprotein to another molecule, comprising:
providing a sample containing a glycoprotein;
treating the sample with a glycosidase to remove a target carbohydrate on the glycoprotein, thereby creating a vacant glycosylation acceptor site on the glycoprotein;
treating the sample with a glycosyltransferase to incorporate a replacement carbohydrate into the vacant glycosylation acceptor site, wherein the replacement carbohydrate includes a click chemistry moiety; and
adding a molecule to the sample, wherein the molecule includes a click chemistry moiety that reacts to the click chemistry moiety of the replacement carbohydrate such that the molecule clickably attaches to the replacement carbohydrate.

20. The method of claim 19 wherein the molecule includes an antibody or a drug.
